(19) United States
(12) Patent Application Publication
(10) Pub. No.: US 2018/0095086 A1
(43) Pub. Date: Apr. 5, 2018

(54) STANDARD CELL SUSPENSION

(71) Applicant: HITACHI CHEMICAL COMPANY, LTD., Tokyo (JP)

(72) Inventors: Satomi YAGI, Tokyo (JP); Katsuya ENDOU, Tokyo (JP); Masako ONOGAMI, Hitachi-shi (JP)

(21) Appl. No.: 15/284,138

(22) Filed: Oct. 3, 2016

(51) Int. Cl. G01N 33/574

(52) U.S. Cl. CPC G01N 33/57423; G01N 2496/00

(57) ABSTRACT

The present invention relates to a standard cell suspension for confirming the stainability of a treatment solution that differentially stains circulating tumor cells (CTCs) and leucocytes, containing (a) a cell line having an antigen that is specific for human cancer cells and (b) a cell line having an antigen that is specific for human leukocytes, wherein the (a) and (b) components are each a cell line that is fixed by a cell-fixing reagent.

### TECHNICAL FIELD

The present invention relates to a standard cell suspension.

### BACKGROUND

Cancer ranks high in the causes of death around the world. In Japan, 300,000 or more people die of cancer every year: there has been a demand for its early diagnosis and treatment. The people's death from cancer is mostly due to metastasis and recurrence of the cancer. The metastasis and recurrence of the cancer occur when cancer cells pass through blood or lymphatic vessels from a primary lesion and adhere to and infiltrate the vascular walls of a different organ tissue to form a micro-metastasis. Such cancer cells that circulate in a human body through blood and lymphatic vessels have been called “circulating tumor cell(s) (also, referred to as “CTC” hereinafter)”.

Blood contains a lot of blood cell components such as erythrocytes, leukocytes, and platelets, and the number of the blood cell components is estimated around to be 3.5 to 9×10⁹ per one mL of blood. By contrast, because CTCs are present only at a level of a few cells, it is necessary to efficiently capture and detect the CTCs among the blood cell components. Various devices for capturing and detecting CTCs have been studied. Specifically, Japanese Unexamined Patent Publication No. 2011-163830 discloses a micro fluid device which comprises a filter made of a nickel substrate having fine through-holes, an upper member made of polydimethylsilloxane (PDMS) that is provided with a sample inlet, and a lower member that is provided with a sample outlet, respectively above and below the filter.

### SUMMARY

In many devices for capturing and detecting CTCs (also, referred to as “CTC capture device(s)” hereinafter), including the micro fluid device disclosed in Japanese Unexamined Patent Publication No. 2011-163830, a specially adapted treatment solution is allowed to pass through the device, a cell fixation, cell membrane permeabilization and antigen-antibody reaction are carried out to differentially stain CTCs and leucocytes, whereby the CTCs are detected. In cases where stained CTCs are not confirmed when the treatment solution is passed through and stained, there is a conceivable possibility that no CTCs are present in a specimen, whereas there is a concerned possibility that the treatment solution does not satisfy the stainability. In the latter instance, there is the possibility that CTCs in the specimen escape detection, which has a significant influence on the performance of the CTC capture device. However, there has not been any known method for confirming the stainability of those treatment solutions.

Thus, an object of the present invention is to provide a standard cell suspension for confirming the stainability of a treatment solution that differentially stains CTCs and leucocytes.

The present invention provides a standard cell suspension for confirming the stainability of a treatment solution that differentially stains circulating tumor cells (CTCs) and leucocytes, comprising (a) a cell line having an antigen that is specific for human cancer cells and (b) a cell line having an antigen that is specific for human leukocytes, wherein the (a) and (b) components are each a cell line that is fixed by a cell-fixing reagent.

If it is possible to respectively confirm CTCs and leucocytes when a treatment solution that differentially stains CTCs and leucocytes is used for detection against the standard cell suspension of the present invention, it can be confirmed that the treatment solution satisfies the stainability. Also, if it is possible to respectively confirm CTCs and leucocytes when the standard cell suspension of the present invention is passed through CTC capture devices and to have (a) and (b) components captured on filters in the CTC capture devices and when a treatment solution that is specifically adapted to each CTC capture device is used to carry out a cell fixation, cell membrane permabilization, and antigen-antibody reaction, it can be confirmed that the treatment solution satisfies the stainability.

In the standard cell suspension, the (a) component may be a human cancer cell line expressing cytokeratin. Cytokeratin is an intermediate filament forming protein that is expressed in epithelial cells and epithelium-derived cells, and it is utilized at a high frequency as a target antigen in detecting CTCs. Therefore, it will be possible to provide a standard cell suspension that is highly versatile.

In the standard cell suspension, the (a) component may be a human cancer cell line expressing a cancer-associated protein. The cancer-associated protein is utilized at a high frequency as a target antigen in detecting CTCs. Therefore, it will be possible to provide a standard cell suspension that is highly versatile.

The cancer-associated protein may be at least one selected from the group consisting of PD-L1, HER2, EGFR, EML4/ALK and VEGFR.

In the standard cell suspension, the (a) component may be a human cancer cell line having a protein that is expressed during epithelial-mesenchymal transformation. The protein that is expressed during the epithelial-mesenchymal transformation is utilized at a high frequency as a target antigen in detecting CTCs. Therefore, it will be possible to provide a standard cell suspension that is highly versatile.

The protein that is expressed during the epithelial-mesenchymal transformation may be at least one selected from the group consisting of Vimentin and E-Cadherin.

In the standard cell suspension, the (b) component may be a human leukemic cell line expressing CD45. CD45 is a protein that is expressed in the entire hematopoietic stem cells, and it is utilized at a high frequency as a target antigen in detecting leukocytes. Therefore, it will be possible to provide a standard cell suspension that is highly versatile.

The standard cell suspension may further comprise (c) adhesion inhibitor. This improves the stabilities of the (a) and (b) components, and the storage stability of the standard cell suspension is improved.

The (c) component may be at least one selected from the group consisting of bovine serum albumin (BSA), polymethacryloyloxyethyl phosphatidylcholin (PMPC), and polyethylene glycol (PEG). Use of such adhesion inhibitor improves the stabilities of the (a) and (b) components more, and the storage stability of the standard cell suspension is improved more.

Further in the standard cell suspension, the (c) component may be BSA and the concentration of BSA may be from 0.1% by mass to 5.0% by mass relative to the total mass of the standard cell suspension. This improves the stabilities of the (a) and (b) components further more, and the storage stability of the standard cell suspension is improved further more.

The present invention also provides a method for confirming the stainability of a circulating tumor cell (CTC) capture device which comprises the steps of: allowing a standard cell suspension to pass through the CTC capture device to capture at least a part of cells in the standard cell suspension; and staining the captured cells with a treatment solution for antigen-antibody reaction, wherein the standard cell suspension comprises (a) a cell line having an antigen that is specific for human cancer cells and (b) a cell line having an antigen that is specific for human leukocytes.

In the method for confirming the stainability of a CTC capture device, the (a) component in the standard cell suspension may be a circulating tumor cell and the (b) component may be leukocyte. Further, in the method for confirming the stainability of a CTC capture device, the (a) component in the standard cell suspension may be a circulating tumor cell and the (b) component may be a cell line having an antigen that is specific for human leukocytes.

The standard cell suspension of the present invention allows the stainability of a treatment solution that differentially stains CTCs and leukocytes to be confirmed. By confirming the stainability of the treatment solution, it will be possible to realize diagnosis with higher precision as well as to prevent false diagnosis such as CTCs having been overlooked.

### DETAILED DESCRIPTION

Embodiments for carrying out the present invention (hereinafter referred to as “the present embodiment”) will concretely be described below. Further, the present invention is not to be limited to the embodiments below and can be practiced in various modifications within the scope of its gist. Still further, the numerical range that is represented by “-” in the present specification embraces each of upper limit and lower limit within its scope.

The standard cell suspension according to the present embodiment is intended for confirming the stainability of a treatment solution that differentially stains CTCs and leukocytes. For example, the standard cell suspension is intended for confirming the stainability of a treatment solution that is used when a CTC capture device is used to separate CTCs in blood and to differentially stain CTCs and residual leukocytes. Specifically, the standard cell suspension is intended for confirming the stainability of a treatment solution that is used when a CTC capture device is used to separate CTCs in blood and to differentially stain CTCs and residual leukocytes with the aid of an antibody against a CTC-specific antigen and an antibody against a leukocyte-specific antigen.

The CTC capture devices include (1) a filter system and (2) a centrifugation system among others. As the filter system of (1), there is mentioned such a micro-cavity array method that is disclosed in Japanese Unexamined Patent Publication No. 2011-163830. According to the micro-cavity array method, the blood from a cancer patient is allowed to flow at a constant speed in a cartridge that retains a filter having a predetermined pore size, and the cell fixation, cell membrane permeabilization, and antigen-antibody reaction are carried out within the device in the semi-automated treatment to detect CTCs. In so doing, an attached treatment solution is used. However, there is no means to conveniently confirm the stainability of the attached treatment solution. Thus, when no CTCs are detected in the patient's blood, it cannot be determined as to whether no CTCs are present in the blood or the deterioration of the treatment solution is the cause, which leads to the possibility of false diagnosis.

The standard cell suspension according to the present embodiment is used to confirm the stainability of the treatment solution for the purpose of preventing the aforementioned false diagnosis.

As the treatment solution, there may be mentioned a treatment solution for cell fixation, a treatment solution for cell membrane permeabilization, and a treatment solution for antigen-antibody reaction, for example.

The treatment solution for cell fixation is a treatment solution for fixing cells so that the degradation of the cells such as their decay or aggregation may be prevented. Known products can be used as the treatment solution for cell fixation, and their examples include the products obtained by dissolving in buffer solutions, cell-fixing reagent such as formaldehyde, parafouualdehyde (PFA), and glutaraldehyde (GA), in addition to methanol.

The treatment solution for cell membrane permeabilization is a treatment solution for partially disrupting the cell membrane so that intracellular molecules may be stained with antibodies or the like. Known products can be used as the treatment solution for cell membrane permeabilization, and their examples include surfactants such as Triton X-100, NP-40, Tween 20; and organic solvents such as methanol that is cooled to about −20° C.

The treatment solution for antigen-antibody reaction is a treatment solution which contains antibodies recognizing antigens that are respectively specific for CTCs and leukocytes and which are intended to detect CTCs and leukocytes each with the attachment of different labels (e.g., fluorescent substances having differing fluorescent wavelengths). The labels may be directly bonded to the respective antibodies; or for example, the labels may be bonded to secondary antibodies that specifically bind to the respective antibodies.

The standard cell suspension according to the present embodiment comprises (a) a cell line having an antigen that is specific for human cancer cells and (b) a cell line having an antigen that is specific for human leukocytes, wherein the (a) and (b) components are fixed by a cell-fixing reagent.

The antigen that is specific for the human cancer cells is, for example, the same antigen as an antigen that is recognized by the antibody specifically binding to CTCs contained in the treatment solution as the object to be confirmed for its stainability. As the example of such antigen, there is mentioned an antigen that is recognized by the labeled antibody specifically binding to CTCs which is used in a CTC capture device. As specific examples of the antigen, there are mentioned antigens that are expressed in epithelial and epithelium-derived cells, including cytokeratin, epithelial cell adhesion molecule (EpCAM), CD146, and CD176. Other specific examples of the antigen include cancer-associated proteins such as PD-L1, HER2, EGFR, EML4/ALK and VEGFR, and proteins that are expressed during the epithelial-mesenchymal transformation, such as Vimentin and E-Cadherin. Because of being utilized at a high frequency as the target antigen in detecting CTCs, the antigen may be cytokeratin, PD-L1, HER2, EGFR, EML4/ALK, VEGFR, Vimentin, and E-Cadherin. Cytokeratin is one of the intermediate filament forming proteins that are expressed in epithelial and epithelium-derived cells.

As the (a) cell line having an antigen that is specific for human cancer cells, there are, for example, mentioned a human cancer cell line expressing the aforementioned antigen and a cell line that is genetically engineered to express the aforementioned antigen. As the human cancer cell line that expresses cytokeratin, epithelial-type cancer cell lines are mentioned. More specifically, there are mentioned the cell lines of lung cancer, breast cancer, colorectal cancer, and stomach cancer. Examples of such cell lines include the non-small cell lung cancer derived cell line NCI-H358 and the human breast cancer cell line SK-BR-3.

When CTCs in blood are to be separated, it is difficult to avoid the contamination of leukocytes that are abundantly contained in the blood. For example, there remains on the filter in a CTC capture device of the filter system, a part of leukocytes having cell sizes that are close to those of CTCs. Therefore, it is necessary to differentially stain the leukocytes and to detect them. The antigen that is specific for human leukocytes is, for example, the same antigen as an antigen that is recognized by the antibody specifically binding to the leukocytes contained in the treatment solution as the object to be confirmed for its stainability. As the example of such antigen, there is mentioned an antigen that is recognized by a labeled antibody specifically binding to the leukocytes and that is used in the CTC capture device. Because of being utilized at a high frequency as the target antigen in detecting leukocytes, the antigen may be CD45. CD45 is antigen that is expressed in the entire hematopoietic stem cells.

As the (b) cell line having an antigen that is specific for human leukocytes, there are, for example, mentioned a human cancer cell line expressing the aforementioned antigen and a cell line that is genetically engineered to express the aforementioned antigen. The (b) cell line having an antigen that is specific for human leukocytes may be a human leukemia cell line, which may be a human leukemia cell line expressing CD45. Examples of such cell lines include the human acute T cell leukemia cell derived cell line Jurkat and the acute lymphocytic leukemia cell derived cell line HAL-01.

It is preferred that the cell lines of the (a) and (b) components are fixed by being subjected to the cell membrane fixation. The case where the cells are not fixed causes the decay or aggregation of the cell lines, which will make the confirmation test of the stainability of reagents difficult.

The fixation of the (a) and (b) components can be carried out by a known method. Examples of the cell-fixing reagent include formaldehyde, paraformaldehyde (PFA), glutaraldehyde (GA), and methanol. More specific example of the cell-fixing reagent includes a solution of PFA dissolved in a phosphate buffer solution (PBS). The PFA concentration of the PFA solution may be from 0.1% by mass to 4.0% by mass.

Mixing of the (a) and (b) components can be carried out after the fixation, for example.

The ratio of (a) component cell number to (b) component cell number in the standard cell suspension according to the present embodiment may be (a) component:(b) component=1:1 or (a) component:(b) component=1:2 because the detection sensitivity is far more superior.

Even if the cell density in the standard cell suspension according to the present embodiment is one cell/mL for both of the (a) and (b) components, it is possible to confirm the stainability of the treatment solution. Since the detection sensitivity can be far more superior, the cell density may be 100 cells/mL-100,000 cells/mL, 500 cells/mL-10,000 cells/mL, or 1,000 cells/mL-2,000 cells/mL.

The standard cell suspension according to the present embodiment may further comprise (c) adhesion inhibitor. The (c) adhesion inhibitor suppresses the adhesion or aggregation among the (a) component, the (b) component, and the container in which the standard cell suspension are filled. If the (c) adhesion inhibitor is contained, the stabilities of the (a) and (b) components improve more, and the storage stability of the standard cell suspension is improved more. Examples of the (c) adhesion inhibitor include bovine serum albumin (BSA), polymethacryloyloxyethyl phosphatidylcholin (PMPC), and polyethylene glycol (PEG). When BSA is used as the (c) component, the concentration of BSA may be 0.1% by mass-5.0% by mass, 0.5% by mass-5.0% by mass, or 0.5% by mass-2.0% by mass relative of the total mass of the standard cell suspension.

Then, the method for confirming the stainability of a treatment solution that differentially stains CTCs and leukocytes by the use of the standard cell suspension according to the present embodiment will be described by taking an example of the case where a CTC capture device of the filter system is used. First, the standard cell suspension according to the present embodiment is allowed to pass through the CTC capture device, and the (a) and (b) component cells are captured on the filter in the CTC capture device. The captured cells are subjected to washing treatment with a buffer solution such as PBS, and the treatment solution for cell fixation is used to fix the cells. Next, the permeabilization of the fixed cells is carried out with the treatment solution for cell membrane permeabilization. Subsequently, the permeated cells are subjected to cell-staining with the treatment solution for antigen-antibody reaction. Antibodies that specifically bind to the (a) and (b) components have respectively different labels (e.g., fluorescent substances with differing wavelengths), for example. The (a) and (b) components that are captured on the filter are observed with a fluorescent microscope; and if it can be confirmed that they are respectively stained, it can be ascertained that the stainability of the treatment solution that has been used to differentially stain CTCs and leukocytes is satisfactory.

The total cell number of the (a) and (b) components that are provided for the CTC capture device may be 100 cells-100,000 cells, 500 cells-10,000 cells, or 1,000 cells-2,000 cells. When the cell number in the standard cell suspension is within such a range, there will be a high possibility that both of the (a) component cell line and the (b) component cell line can be detected.

The present invention also provides a method for confirming the stainability of a CTC capture device. The method for confirming the stainability of a CTC capture device according to the present embodiment comprises the steps of: allowing a standard cell suspension to pass through the CTC capture device to capture at least a part of cells in the standard cell suspension; and staining the captured cells with a treatment solution for antigen-antibody reaction, wherein the standard cell suspension comprises (a) a cell line having an antigen that is specific for human cancer cells and (b) a cell line having an antigen that is specific for human leucocyte. If it can be confirmed that the cells that have been captured with the treatment solution for antigen-antibody reaction are stained, it can be ascertained that the stainability of the CTC capture device is satisfactory.

In the method for confirming the stainability of a CTC capture device according to the present embodiment, the (a) component may be a circulating tumor cell and the (b) component may be leukocyte.

In the method for confirming the stainability of a CTC capture device according to the present embodiment, the standard cell suspension may be the standard cell suspension according to the present invention as described above.

The present invention will be described more specifically based on examples hereinafter. Nevertheless, the present invention will not be limited to the examples below.

### Example 1

[Preparation of Standard Cell Suspension Containing Fixed Cell Lines (Studies on the Concentrations of Adhesion Inhibitor)]

Cell line NCI-H358 derived from the non-small cell lung cancer was used as (a) component and cell line Jurkat derived from the human acute T cell leukemia cells was used as (b) component, respectively. Bovine serum albumin (BSA) was used as (c) component to study the conditions of BSA concentrations.

NCI-H358 cells adhered to a flask were subjected to peeling treatment with a 0.25% by mass trypsin-EDTA solution. A centrifuge (manufactured by Hitachi Koki Co., Ltd.: CT15E) was used to carry out centrifugation [1000 rpm (180×g), 3 minutes], and washing with a phosphate buffer solution (PBS) was carried out twice to remove the trypsin-EDTA solution. Subsequently, 4% by mass PFA-PBS was used to carry out the fixation for 2 hours. After reaction, PFA-PBS was removed and washing was carried out with PBS twice. PBS was substituted with two types of the adhesion inhibitor with varying concentrations of BSA, i.e., (1) 0.5% by mass BSA-PBS and (2) 5.0% by mass BSA-PBS, respectively.

Jurkat cells that were floating in a flask were centrifuged at 1,000 rpm (180×g) for 3 minutes and washing was carried out with PBS twice. Subsequently, 4% by mass PFA-PBS was used to carry out the fixation for 2 hours. After reaction, PFA was removed and washing was carried out with PBS twice. PBS was substituted with two types of the adhesion inhibitor with varying concentrations of BSA, i.e., (1) 0.5% by mass BSA-PBS and (2) 5.0% by mass BSA-PBS, respectively.

[Confirmation of Physical Stability of Fixed Cell Lines: Measurement of Particle Sizes of the Cells]

The standard cell suspension after the fixation was stored for a prescribed period, and a CASY Cell Counter (Roche) was used to measure the particle sizes of the cells. The measurement results of the particles sizes of NCI-H358 cells are shown in Table 1; The measurement results of the particles sizes of Jurkat cells are shown in Table 2. The respective cells were subjected to the cell particle size measurement at Day 3, Day 21, and Day 51. Measuring conditions: the inner diameter of a capillary used—150 μm; and sample cell density−5×10⁶ cells/mL.

TABLE 1

| | Cell particle size (μm) | | |
|---|---|---|---|
| Adhesion inhibitor | Day 3 | Day 21 | Day 51 |
| 0.5% by mass BSA-PBS | 17.0 | 15.7 | 14.7 |
| 5.0% by mass BSA-PBS | 16.0 | 13.5 | 12.0 |

TABLE 2

| | Cell particle size (μm) | | |
|---|---|---|---|
| Adhesion inhibitor | Day 3 | Day 21 | Day 51 |
| 0.5% by mass BSA-PBS | 11.8 | 10.7 | 10.1 |
| 5.0% by mass BSA-PBS | 10.6 | 9.5 | 8.8 |

Based on Table 1 and Table 2, the reduction in the cell particle size was smaller in the case of BSA concentration being 0.5% by mass. Thus, it was determined that in order to store the cells more stably, the concentration of BSA as the adhesion inhibitor was preferably 0.5% by mass.

### Example 2

[Continuation of Stainability of Treatment Solution Using Standard Cell Suspension with Fixation]

Mixing preparation was carried out in 0.5% by mass BSA-PBS so that the fixed NCI-H358 cells (Day 3) would reach a final density of 1,000 cells/mL and the fixed Jurkat cells would reach a final density of 2,000 Cells/mL, whereby the standard cell suspension was prepared. The prepared standard cell suspension was treated in a CTC capture device to confirm the stainability of the standard cell suspension. The CTC capture device used was a device where a metal filter having a prescribed pore size (8×30 μm) was provided in a cartridge made of polymethylmethacrylate (PMMA) and where suction could be applied at a prescribed rate from the outlet channel within the cartridge.

The standard cell suspension, 1.0 mL, was allowed to pass through the CTC capture device, and the fixed cells were captured on the filter within the cartridge. Subsequently, the cells were subjected to the washing treatment with PBS, the fixation with 4% by mass PFA-PBS for 20 minutes, and the permeabilization with a solution of 0.2% by mass Triton X-100 (registered trademark; Sigma-Aldrich Co. LLC). Next, the antibody staining treatment was carried out by using a staining solution at 25° C. for 30 minutes, and the stainability of the reagents was confirmed by the stained degrees of the cells captured on the filter. The staining solution employed Anti-Pan-Cytokeratin (AE1/AE3) Alexa Fluor 488 as an anti-cytokeratin antibody, Anti-Human CD45 PE as an anti-CD45 antibody, and Hoechst 33342 reagent as a cell nucleus staining reagent.

The NCI-H358 cells were detected in the wavelength region of FITC (522 nm); The Jurkat cells were detected in the wavelength region of PE (578 nm). It was ascertained that these cells are adequate to confirming the staining. Also, both of the NCI-H358 cells and the Jurkat cells were detected in the wavelength region (461 nm) of Hoechst 33342 as the cell nucleus staining reagent. Moreover, the NCI-H358 cells were not detected in the wavelength region of PE; The Jurkat cells were not detected in the wavelength region of FITC. This demonstrated that the stainability of the treatment solutions can be confirmed by using the standard cell suspension according to the present embodiment.

1. A standard cell suspension for confirming the stainability of a treatment solution that differentially stains circulating tumor cells (CTCs) and leucocytes, comprising:
(a) a cell line having an antigen that is specific for human cancer cells; and
(b) a cell line having an antigen that is specific for human leukocytes,
wherein the (a) and (b) components are each a cell line that is fixed by a cell-fixing reagent.

2. The standard cell suspension according to claim 1, wherein the (a) component is a human cancer cell line expressing cytokeratin.

3. The standard cell suspension according to claim 1, wherein the (a) component is a human cancer cell line expressing a cancer-associated protein.

4. The standard cell suspension according to claim 3, wherein the cancer-associated protein is at least one selected from the group consisting of PD-L1, HER2, EGFR, EML4/ALK, and VEGFR.

5. The standard cell suspension according to claim 1, wherein the (a) component is a human cancer cell line having a protein that is expressed during epithelial-mesenchymal transformation.

6. The standard cell suspension according to claim 5, wherein the protein that is expressed during the epithelial-mesenchymal transformation is selected from the group consisting of Vimentin and E-Cadherin.

7. The standard cell suspension according to claim 1, wherein the (b) component is a human-derived cell line expressing CD45.

8. The standard cell suspension according to claim 1, further comprising (c) adhesion inhibitor.

9. The standard cell suspension according to claim 8, wherein the (c) component is at least one selected from the group consisting of bovine serum albumin (BSA), polymethacryloyloxyethyl phosphatidylcholin (PMPC), and polyethylene glycol (PEG).

10. The standard cell suspension according to claim 8, wherein the (c) component is BSA and the concentration of BSA is from 0.1% by mass to 5.0% by mass relative to the total mass of the standard cell suspension.

11. A method for confirming the stainability of a circulating tumor cell (CTC) capture device, comprising the steps of:
allowing a standard cell suspension to pass through the CTC capture device to capture at least a part of cells in the standard cell suspension; and
staining the captured cells with a treatment solution for antigen-antibody reaction,
wherein the standard cell suspension comprises (a) a cell line having an antigen that is specific for human cancers cell and (b) a cell line having an antigen that is specific for human leukocytes.

12. The method for confirming the stainability of a CTC capture device according to claim 11, wherein the (a) component is a circulating tumor cell and the (b) component is a leukocyte.

13. The method for confirming the stainability of a CTC capture device according to claim 11, wherein the (a) component is a circulating tumor cell and the (b) component is a cell line having an antigen that is specific for human leukocytes.
